(19) United States
(12) Patent Application Publication
(10) Pub. No.: US 2018/0185135 A1
(43) Pub. Date: Jul. 5, 2018

(54) IMPLANTABLE HOLDING DEVICE

(71) Applicant: Meister & Cie AG Hasle-Rüegsau, Hasle-Ruegsau (CH)

(72) Inventors: Marcel MEISTER, Hasle-Ruegsau (CH); Philippe GEDET, Ipsach (CH)

(21) Appl. No.: 15/740,071

(22) PCT Filed: Jun. 15, 2016
(86) PCT No.: PCT/EP2016/063798

§ 371 Date: Dec. 27, 2017

(30) Foreign Application Priority Data
Jul. 24, 2015 (EP) 15178204.2

(51) Int. Cl. A61F 2/08; A61B 17/04

(52) U.S. Cl. CPC A61F 2/0811; A61B 17/0401; A61B 2017/0404; A61F 2002/0823; A61B 2017/0464; A61F 2002/0882; A61F 2002/0852; A61B 2017/0414

(57) ABSTRACT

The Implantable holding device for acting on bones, in particular for ligament reconstruction and/or stabilization of a joint, includes a flexible loop member having a first end, a second end and a plurality of eyelets, and a support member configured to lie against a bone and including an attachment element. The loop member being braided to form bifurcating and converging portions which define the eyelets. The support member has a first attachment location for attachment of the first end of the loop member and a second attachment location for attaching the loop member by passing a portion of the attachment element through an eyelet.

The invention relates to a holding device comprising a flexible member and a support member configured to lie against a bone.

Such holding devices are for instance used for ligament reconstruction. A ligament, such as the anterior cruciate ligament (ACL) or posterior cruciate ligament (PCL), which has ruptured in a non-repairable way, may be replaced arthroscopically by a tissue graft, which may be harvested form another portion of the body or which may be artificial. The holding device is used to attach the tissue graft to the bone, e.g. the femur.

One holding device available on the market is sold under the name RetroButton®. This product comes in a set of titanium buttons with various sizes and continuous loops with different length. During operation, the surgeon chooses out of the set that button which has the loop with the appropriate size so that the tissue graft can be attached with the desired strength. However, the provision of various sizes makes manufacturing as well as storage and handling at the consumer's side complicated.

The patent application EP 2 476 378 A1 describes a holding device in form of an anchor comprising an anchor body, a continuous closed suture loop and an insert. For assembling the anchor a portion of the suture loop is passed through an opening in the anchor body and secured by the insert. The size of the loop is fixed. This has the disadvantage mentioned above that several anchors with different sizes have to be provided, out of which the surgeon chooses an anchor which is suitable to attach a specific tissue graft with the desired strength.

The patent application WO 2009/036286 A1 relates to a device of a kind that is different from the holding devices mentioned above. There is provided an anchor block having a knob for engagement of one end of a tension member. The other end of the tension member is configured for an engagement with a tissue. No flexible member is provided over which e.g. a tissue graft can be looped for attachment.

The patent application US 2013/0116787 A1 discloses a holding device comprising a support member and a ratchet mechanism, which is formed of solid members and which serves for adjusting the length. The design of this holding device is relatively complicated. Particular measures have to be taken in order to avoid a loosening of the ratchet mechanism, once it is implanted.

It is an aim of the present invention to provide for an improved holding device that it is adjustable in size.

This aim is achieved by the holding device as defined in claims 1. The further claims specify preferred embodiments of the device.

There is provided a support member having two attachment locations for attachment of the ends of a flexible loop member. The latter has a plurality of eyelets, through which a portion of an attachment element of the support member can be passed. This has the advantage that the effective length of the loop member, i.e. the length, which the loop member has between the two attachment locations, is adjustable by choosing a specific eyelet for the attachment. The end part of the loop member which is too long may be cut away. The eyelets allow also a simplified handling of the holding device by passing e.g. an auxiliary suture therethrough for shuttling the holding device through a bore.

Providing the loop member as a braid results in a structure having a high strength, in particular at the bifurcating and converging locations of the eyelets.

Preferably, the holding device has one or more of the following features:
The flexible loop member is configured as an elongated structure, which extends from the first end to the second end.
The support member and the loop member are configured such that the loop member, when attached at the two attachment locations, form together with the support member a loop through which e.g. a graft, a bone portion or another tissue portion to be attached may pass.
The flexible loop member has more than two eyelets.
The loop member is braided to form alternately bifurcating and converging portions which define the eyelets.
The eyelets are formed in that, in an alternating manner, a central portion of the loop member bifurcates into separated branch portions, which converge into an adjacent central portion.

Additional features and their advantages will be become apparent from the following description of exemplary embodiments and from the drawings, where

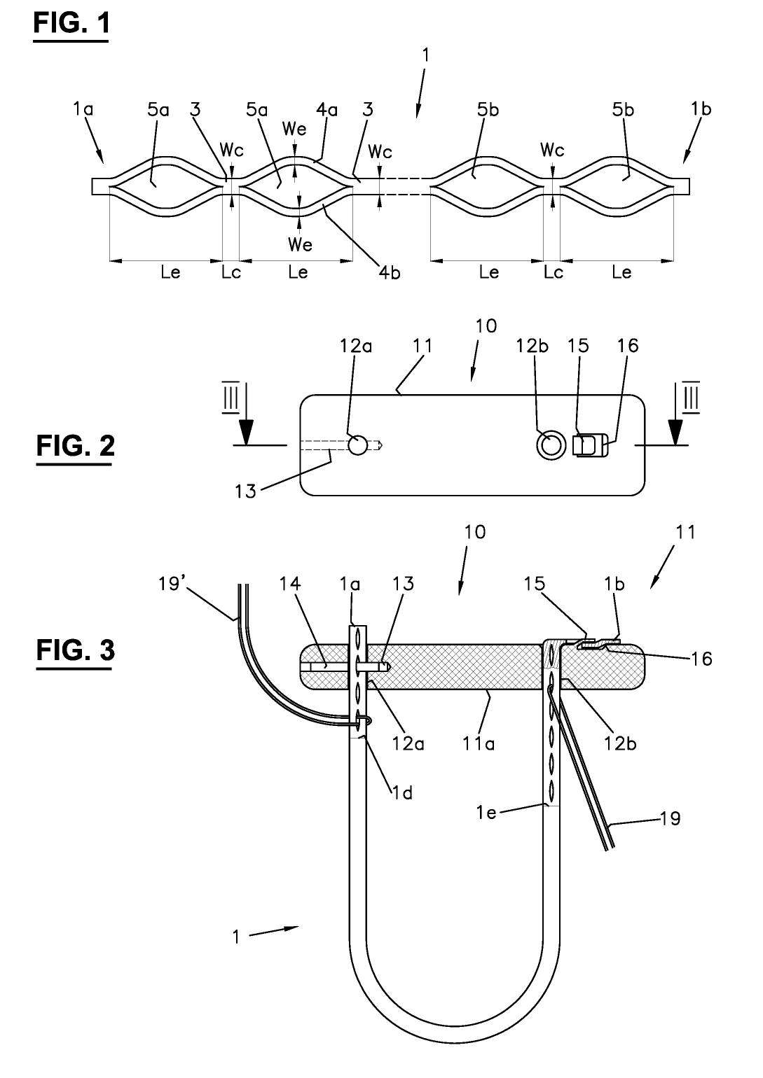

FIG. 1 is a schematic view of a loop member, wherein only the two ends are shown and the intermediate portion is indicated by dashed lines;

FIG. 2 shows a top view of a support member for forming a first embodiment of a holding device;

FIG. 3 shows the loop member of FIG. 1 and the support member sectioned along line III-III in FIG. 2, wherein also an auxiliary suture is shown;

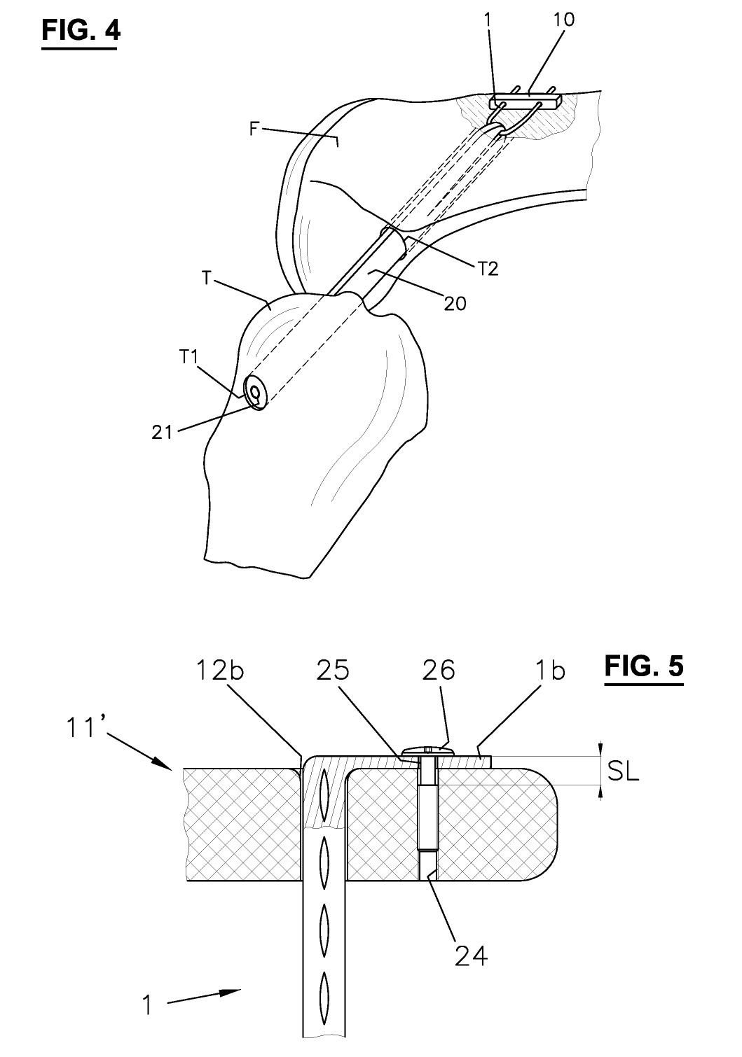

FIG. 4 shows a view of femur and tibia and the holding device of the first embodiment together with a tissue graft attached thereto;

FIG. 5 shows a variant of the left attachment location of the holding device of FIG. 3;

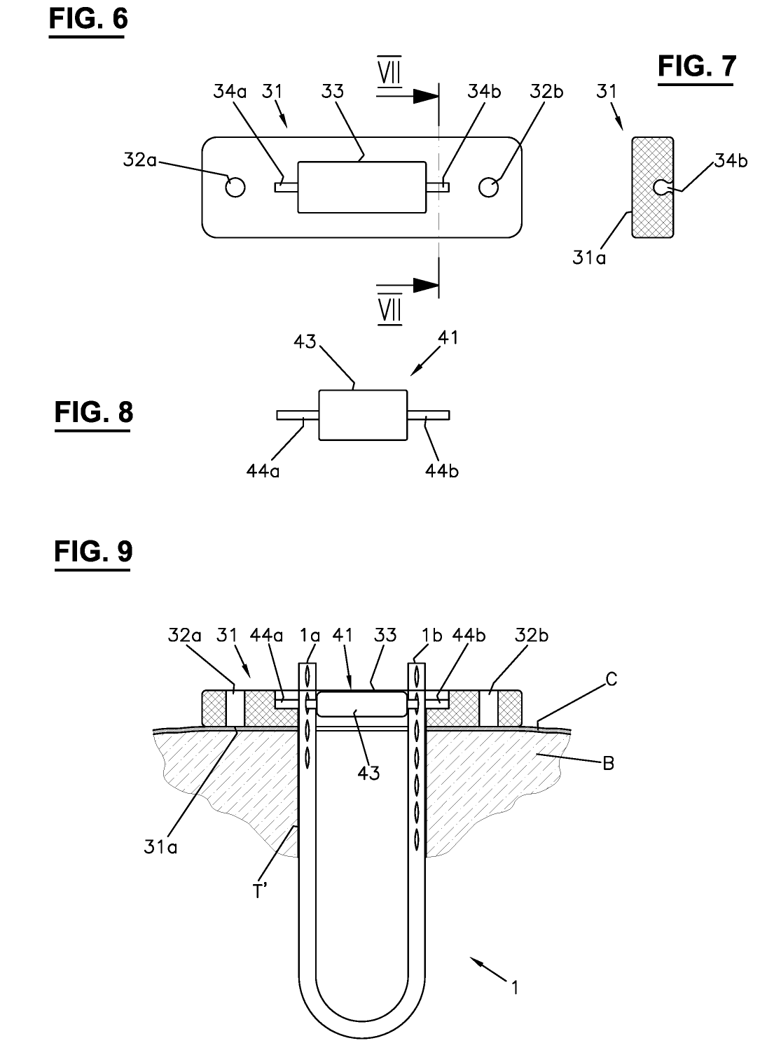

FIG. 6 shows a top view of a base element for forming a second embodiment of a holding device;

FIG. 7 shows the base element sectioned along line VI-VI in FIG. 6;

FIG. 8 shows a top view of an attachment element for forming a second embodiment of a holding device;

FIG. 9 shows the elements of FIGS. 6 and 8 together with the loop member of FIG. 1 in a partially sectioned side view;

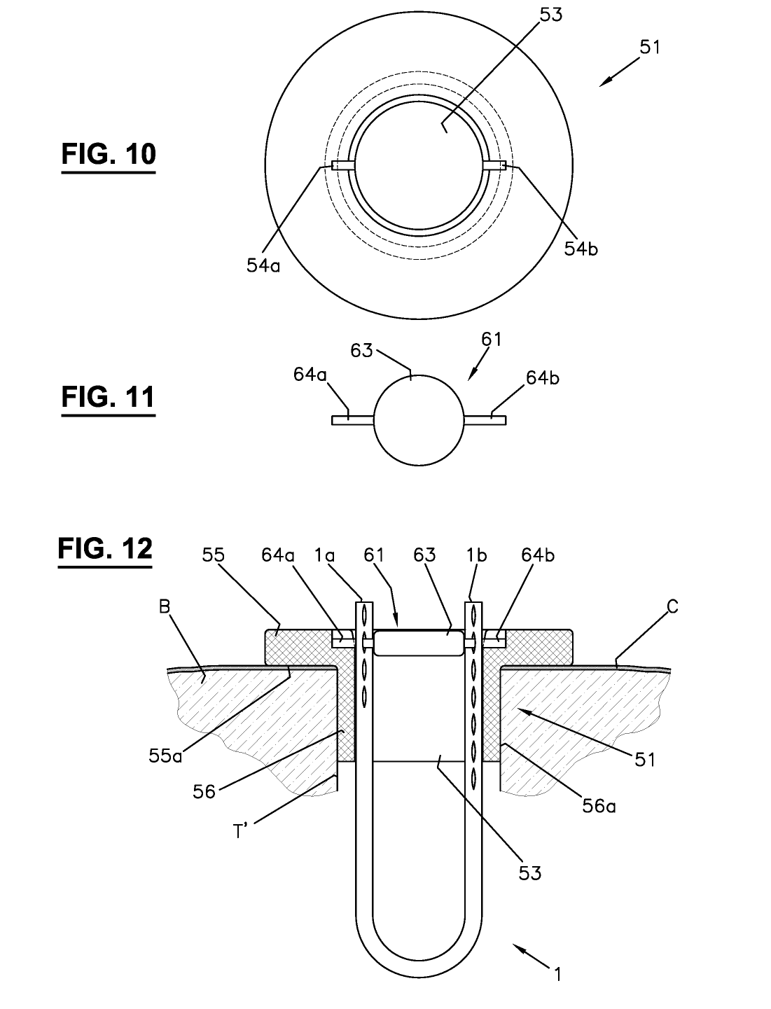

FIG. 10 shows a top view of a base element for forming a third embodiment of a holding device;

FIG. 11 shows a top view of an attachment element for forming the third embodiment of a holding device;

FIG. 12 shows the elements of FIGS. 10 and 11 together with the loop member of FIG. 1 in a partially sectioned side view;

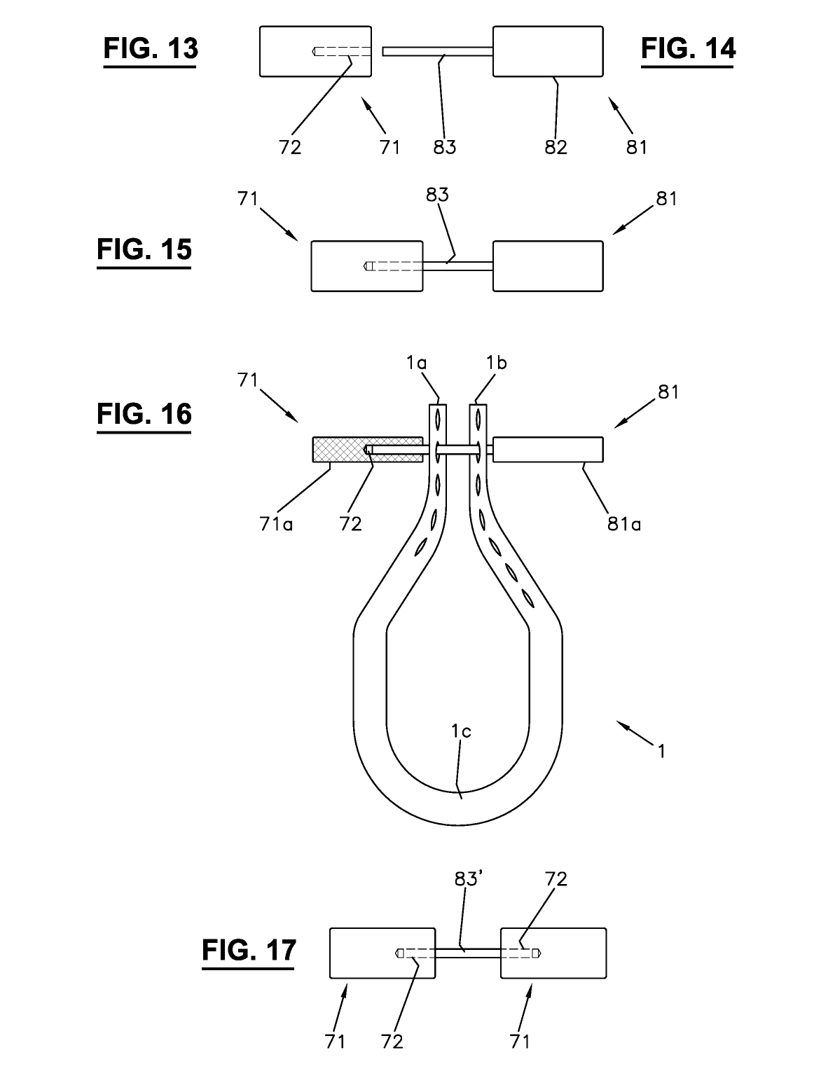

FIG. 13 shows a top view of a base element for forming a fourth embodiment of a holding device;

FIG. 14 shows a top view of an attachment element for forming the fourth embodiment of a holding device;

FIG. 15 shows a top view of the elements of FIGS. 13 and 14 assembled together;

FIG. 16 shows the arrangement of FIG. 15 together with the loop member of FIG. 1 in a partially sectioned side view;

FIG. 17 shows a variant of the arrangement of FIG. 15 in a top view;

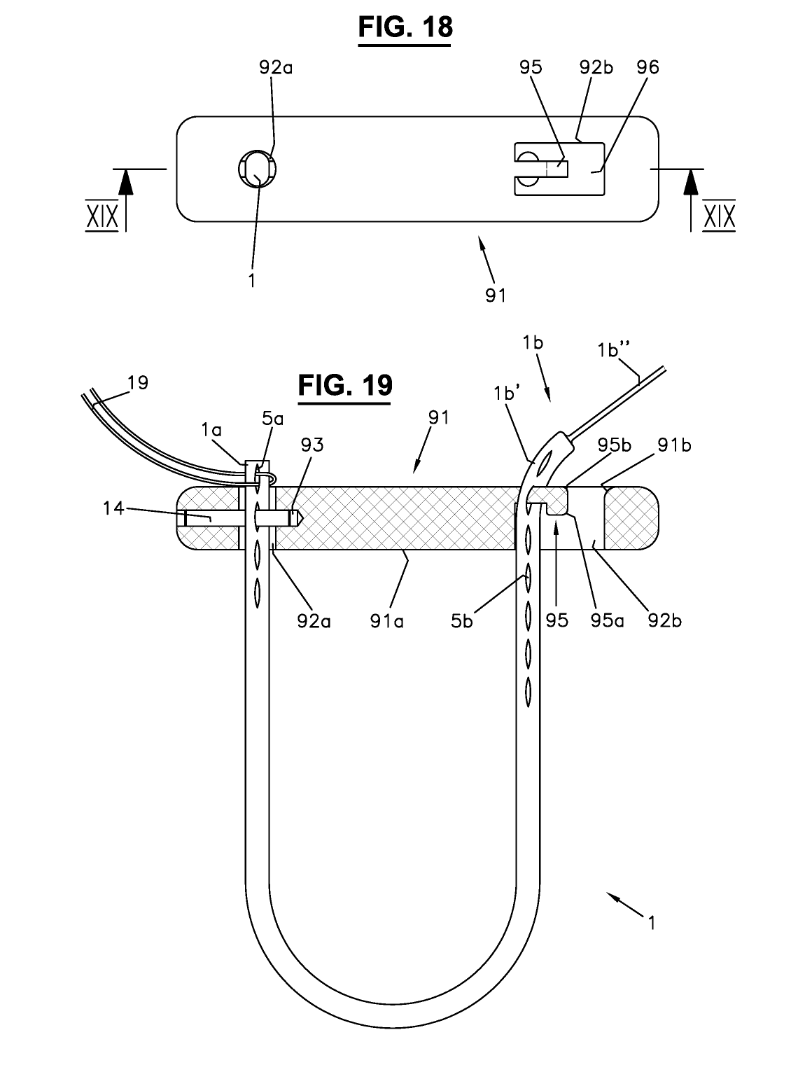

FIG. 18 shows a top view of a support member for forming a fifth embodiment of a holding device, wherein only part of a loop member is shown;

FIG. 19 shows the holding device of FIG. 18 sectioned along the line XIX-XIX, wherein also an auxiliary suture is shown;

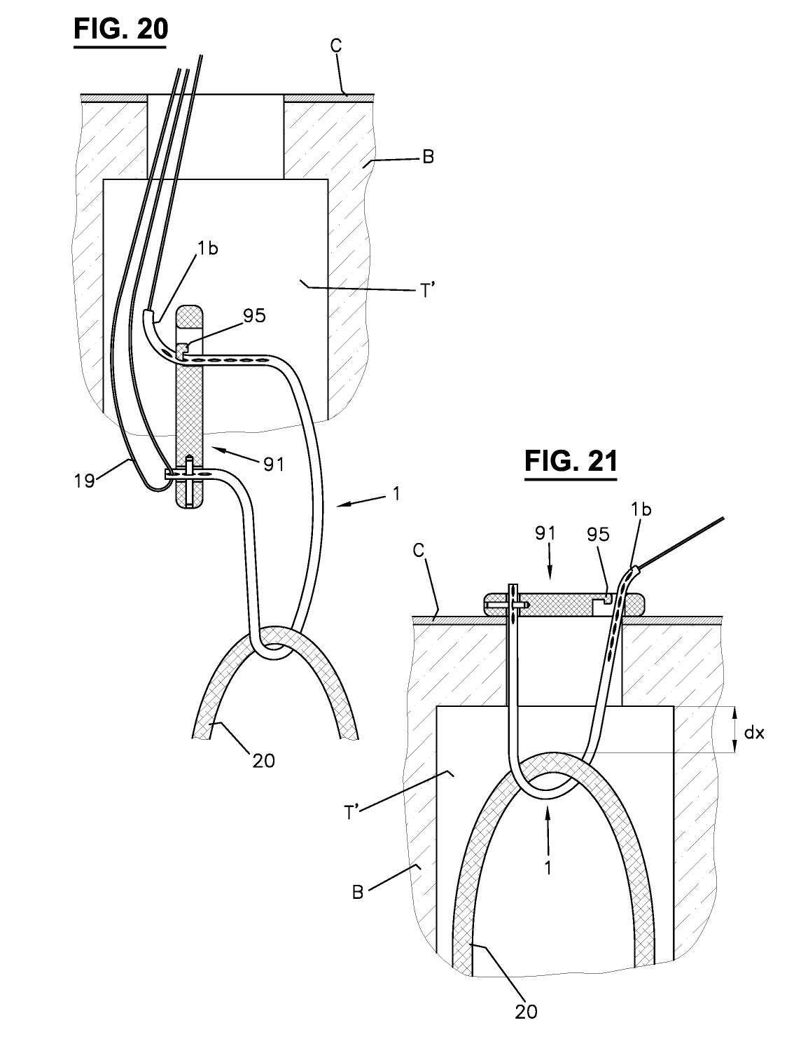

FIG. 20 shows the holding device of FIG. 19 used for attaching a tissue graft to a bone;

FIG. 21 shows the arrangement of FIG. 20, wherein the support member abuts on the bone surface and the tension of the tissue graft is adjusted by pulling on the loop member;

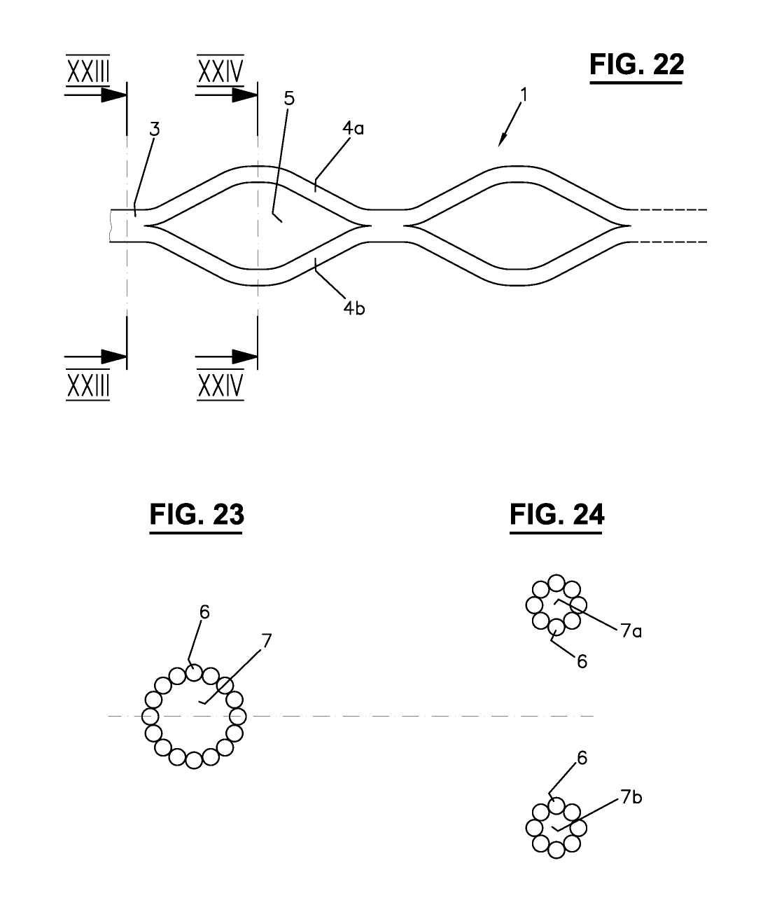

FIG. 22 shows a detailed view of the loop member of FIG. 1;

FIG. 23 shows the loop member sectioned XXIII-XXIII in FIG. 22; and

FIG. 24 shows the loop member sectioned XXIV-XXIV in FIG. 22.

FIG. 1 shows a loop member 1 to be used together with a support member to provide a holding device. The loop member 1 is formed as a flexible slender structure, which extends from a first end 1a to a second end 1b and which includes a multiple of eyelets 5a, 5b. An eyelet 5a, 5b is formed in that a central portion 3 of the loop member 1 bifurcates into separated branch portions 4a, 4b, which again merges into a central portion 3. The number of eyelets 5b at the second end 1b is more than two.

The length Le of the eyelets 5a, 5b may be the same or vary. The length Lc of the central portions 3 may be the same or vary. For instance the length Lc of the central portions 3, which are near the first end 1a, may be different to the length Lc of the central portions 3, which are near to the second end 1b. This increases the possibilities of choosing a particular length between the first end 1a and an eyelet 5b near the second end 1b. In one embodiment, the loop member 1 has an intermediate portion which is arranged between the ends 1a, 1b and which is free of eyelets 5a, 5b. The length of this intermediate portion may be greater than the length of the end 1a with the eyelets 5a and/or the length of the end 1b with the eyelets 5b.

The width Wc of the central portions 3 may be the same or vary and the width We of a branch portion 4a, 4b may be the same or vary. In one embodiment, the width Wc of each central portion 3 is substantially constant and/or approximately twice the width We. In another embodiment, the loop member 1 has an intermediate portion which is arranged between the ends 1a and 1b and which is greater in thickness Wc than the ends 1a and 1b. Thereby, a particular large supporting surface to support a graft tissue can be provided.

FIGS. 2 and 3 show a first embodiment of a holding device including a support member 10 and a loop member 1. The support member 10 comprises a plate 11 having a flat bottom side 11a to be contacted to a bone of a patient and two through-going windows 12a, 12b which are adapted to pass therethrough the ends 1a, 1b of the loop member 1.

Preferably, the plate 11 has a form which is free of sharp areas by rounding off e.g. the corners and/or edges. The plate 11 comprises a blind hole 13 which extends from a lateral side of the plate 11 through the first window 12a and which is adapted to receive a fixation pin 14. The diameter of the blind hole 13 is configured such that the fixation pin 14 may be press-fitted therein.

An attachment mean 15 to attach the second end 1b of the loop member 1 is arranged adjacent to the second window 12b. Here, the attachment mean is a tongue 15 which is preferably made integral with the plate 11 and which may be configured such that it is rigid or bendable. The plate 11 may comprise a recess 16 opposite to the tongue 15 to receive part of the loop member 1.

The holding device may be preassembled such that the first end 1a of the loop member 1 is already fed through the window 12a and fixed by passing the fixation pin 14 through an eyelet of the loop member 1, whereas the second end 1b of the loop member 1 is loose. The holding device may be sterilized and packed.

The configuration of the loop member 1 is adapted to the desired usage of the holding device. For instance, the loop member 1 may be configured such that eyelets are provided near the ends 1a, 1b, whereas the intermediate portion, which is indicated in FIG. 3 as the portion between the locations 1d and 1e, has some eyelets or is free of eyelets. The length of the loop member between the locations 1d and 1e may be larger than the length between the first end 1a and the location 1d and/or the length between the second end 1b and the location 1e. It is also conceivable to configure the loop member 1 such that its width is substantially constant or such that the intermediate portion of the loop member 1 is greater in thickness than the ends 1a, 1b.

The holding device is for instance usable to reconstruct an anterior cruciate ligament as shown in FIG. 4. During surgery, the surgeon drills a tunnel T1, T2 through the femur F and the tibia T. The tissue graft 20, which is used to replace the ACL, e.g. a semitendinosus-tendon graft, is put around the loop member 1. A shuttle suture 19′ (see FIG. 3) is passed through the tunnel T1, T2 and put through an eyelet of the loop member 1 near the first end 1a. By pulling on the shuttle suture 19′ the end of the plate 11 where the first end 1a is attached is moved through the tunnel T1, T2 together with the loop member 10 until the other end of the plate 11 comes out of the tunnel T2 and can be flipped with the flip suture 19 so that the bottom side 11a abuts on the bone surface. Before pulling the plate 11 through the tunnel T1, T2, the second end 1b of the loop member 1 is attached to the support member 11 by means of the attachment mean 15, whereby the surgeon chooses that eyelet 5b of the loop member 1 which results in the desired length of the loop member 1 between the two windows 12a, 12b. The end 1b may be shortened by cutting away the portion which is too long. The end of the tissue graft 20 may be fixed to the tibia T e.g. by means of a screw 21. Finally, the tissue graft 20 is hold by the holding device 1, 10 with the desired strength.

In an alternative usage, it is conceivable to provide a loop member 1 long enough so that the intermediate portion of the loop member 1 only is moved through the tunnel T1, T2 to attach the graft tissue and pulled back again, whereas the support member 1 and the two ends 1a, 1b remain during operation outside the tunnel T2.

Various possibilities are conceivable to attach the second end 1b of the loop member 1 to the support member. FIG. 5 shows a variant, in which the plate 11′ of the support member has a threaded hole 24 to receive a screw 26. The head of the screw 26 includes a drive for cooperation with a driver for rotating the screw 26. The part 25 adjacent to the head of the screw 26 is for the length SL cylindrical before the threaded part starts, which is adapted to engage with the thread of the hole 24. To attach the loop member 1 on the plate 11′, the end 1b is guided through the window 12b and fixed by means of the screw 26, so that it passes through an eyelet 5b and is received in the hole 24.

Another possible embodiment of attaching the second end 1b of the loop member 1 to the support member is described below with regard to FIGS. 18 and 19.

FIGS. 6 to 9 show a second embodiment of a holding device including a support member 31, 41 and a loop member 1. The support member is formed in two pieces: a plate 31 and an attachment element 41 being releasably attachable thereto. The plate 31 comprises a flat bottom side 31a to abut the surface of a bone B. (The layer C in FIG. 9 refers to the cortex, which is the outer shell of the bone B). The plate 31 further includes a window 33, which extends through the wall of the plate 31, and two grooves 34a, 34b, which are arranged in line with each other and which lead to the window 33. The plate 31 may have one or more through-going holes 32a, 32b for handling the holding device e.g. by means of a shuttle suture. The holes 32a, 32b are arranged e.g. in line with the grooves 34a, 34b.

The attachment element 41 includes an intermediate portion 43 and two arms 44a, 44b which are arranged in line with each other and which are attached to the lateral sides of the intermediate portion 43. The grooves 34a, 34b and the arms 44a, 44b are designed to enable a snap engagement. To this end, the cross-section of a groove 34a, 34b may have an undercut so that an arm 44a, 44b when pressed into the corresponding groove 34a, 34b is retained therein.

When using the holding device according to the second embodiment for replacement of a ligament, the surgeon puts the tissue graft around the loop member 1 and guides each arm 44a, 44b of the attachment element 41 through an eyelet of the loop member 1 so that the portion of the loop member 1 between the arms 44a, 44b has the desired length. Subsequently, the attachment element 41 together with the ends 1a, 1b of the loop member 1 is attached to the plate 31 by pressing the arms 44a, 44b into the grooves 34a, 34b. A suture which extends through a tunnel T formed in the bone B is passed through one of holes 32a, 32b of the plate 31. By pulling on the suture the elements 1, 31, 41 can be moved through the tunnel T′ until the support member 31, 41 comes out of the tunnel T′ and can be arranged such that the bottom side 31a abuts the bone surface. In an alternative application, it also possible to pull only the ends 1a, 1b of the loop member 1 through the tunnel T′ and subsequently attach them to the plate 31 by means of the attachment element 43.

FIGS. 10 to 12 show a third embodiment of a holding device including a support member 51, 61 and a loop member 1. The support member includes two pieces: a tubular element 51 and an attachment element 61 being releasably attachable thereto. In the present embodiment, the tubular element 51 has an axially symmetrical form. It comprises a through-going passage 53 to receive the loop member 1. The passage 53 has a substantially circular cross-section. The tubular element 51 includes a flange portion 55 and an insert portion 56. The flange portion 55 has an outer diameter which is greater than the outer diameter of the insert portion 56, so that it extends laterally over insert portion 56. The underside 55a of the flange portion 55 is substantially flat to lie against the surface of a bone B. (The layer C in FIG. 12 refers to the cortex, which is the outer shell of the bone B). The flange portion 55 includes two grooves 54a, 54b which are arranged in line with each other and which end in the passage 53. The insert portion 56 may have an outer surface 56a which is cylindrical. In an alternative embodiment, the outer surface 56a tapers, for instance in a conical manner.

The attachment element 61 includes an intermediate portion 63 and two arms 64a, 64b which are arranged in line with each other and which are attached to the intermediate portion 63. The grooves 54a, 54b and the arms 64a, 64b are designed to enable a snap engagement. To this end, the cross-section of a groove 54a, 54b may have an undercut so that an arm 64a, 64b when pressed into the corresponding groove 54a, 54b is retained therein.

When using the holding device according to the third embodiment for replacement of a ligament, the surgeon drills a boring T′ in the bone B, e.g. the femur. The inlet of the boring T′ may be formed such that it is adapted to the outer surface 56a of the tubular element 51. For instance, the inlet of the boring T′ may be formed cylindrically or conically to receive a cylindrical or conical insert portion 56. The loop member 1 and the graft tissue attached thereto are pulled through the boring T′ by means of a shuttle suture which has been passed through two eyelets in the end 1a and 1b of the loop member 1. Subsequently, each arm 64a, 64b of the attachment element 63 is passed through an eyelet at the end 1a and 1b, respectively, so that the loop member 1 has the desired length therebetween. The attachment element 63 is then attached to the tubular element 51 which is received in the boring T′.

FIGS. 13 to 16 show a fourth embodiment of a holding device including a support member 71, 81 and a loop member 1. The support member includes two pieces: a first plate element 71 and a second plate element 81, which are releasably attachable to each other. The first and second plate elements 71, 81 have a flat bottom side 71a, 81a to be contacted to a bone. The first plate element has a substantially rectangular shape and includes a blind hole 72 extending from a lateral side into the interior. The second plate element 81 has a base body 82, which has a substantially rectangular shape, and an arm 83, which extends laterally from the base body 82. The arm 83 is adapted to be received in the blind hole 72, whose diameter may be chosen such that the arm 83 can be held therein by means of a press fit.

The handling of the holding device is similar to the ones described above. The loop member 1 is attachable to the support member 71, 81 by passing the arm 83 first through an eyelet of each arm 1a, 1b and then into the blind hole 72. FIG. 16 shows a variant of the loop member 1 whose intermediate portion 1c is wider than its ends 1a, 1b. Thereby, a particular large support surface can be provided for holding a tissue graft.

The second plate element 81 may be formed in one or more pieces. A particular simplified variant is to provide a support member including two elements in the form of the first plate element 71 and a pin 83′, which can be pushed into the blind holes 72 of first plate element 71. This variant is shown in FIG. 17.

FIGS. 18 and 19 show a fifth embodiment of a holding device including a support member and a loop member 1, which is in particular suitable to adjust the length of the loop member 1 when the support member is already placed on its final position on the bone.

The support member comprises a plate 91 having a flat bottom side 91a to be contacted to a bone and two through-going windows 92a, 92b which are adapted to pass therethrough the ends 1a, 1b of the loop member 1. Preferably, the plate 91 has a form which is free of sharp areas by rounding off and/or chamfering e.g. the corners and/or edges. The plate 91 comprises a blind hole 93 which extends from a lateral side of the plate 91 through the first window 92a and which is adapted to receive a fixation pin 14. The diameter of the blind hole 93 is configured such that the fixation pin 14 may be press-fitted therein.

An attachment mean 95 to attach the second end 1b of the loop member 1 is arranged in the second window 92b. Here, the attachment mean is a hook 95 which is preferably made integral with the plate 91. The hook 95 extends into the window 92b such that there is a free space 96 between the hook 95 and the walls of the plate 91 which define the window 92b. This space 96 allows the user to pass the end 1b of the loop member 1 through the window 92b, to move it relative to the hook 95 and to put it thereover such that the hook 95 extends through a specific eyelet and the portion of the loop member 1 between the fixation pin 14 and the hook 95 has the desired length.

As is in particular apparent from FIG. 19, the distal edge 95a and proximal edge 95b of the hook 95 may be chamfered and/or rounded. The wall of the plate 91 which defines a side of the window 92b and which is opposed to the forward end of the hook 95 may be chamfered and/or rounded at the upper edge 91b.

The holding device may be preassembled such that the first end 1a of the loop member 1 is already fed through the window 92a and fixed by passing the fixation pin 14 through an eyelet 5a of the loop member 1, whereas the second end 1b of the loop member 1 is loose. The holding device may be sterilized and packed.

In the embodiment shown in FIG. 19 the loop member 1 is provided with a flip suture 19 passed through an eyelet 5a near the first end 1a, either on the bottom side 91a or on the top side of plate 91. The second end 1b of the loop member 1 is formed by an eyelet portion 1b′ which has eyelets 5b and an end portion 1b″ which follows the eyelet portion 1b′ and which is free of eyelets 5b. The end portion 1b″ may be reduced in diameter with respect to the eyelet portion 1b′.

Instead of the end portion 1b″ or in addition thereof the second end 1b of the loop member 1 may be provided with a shuttle suture 19′ similar as shown in FIG. 3, either on the bottom side or on the top side of plate 91.

FIGS. 20 and 21 illustrate a possible application of the holding device according to FIG. 19, wherein a portion of a bone B in which a tunnel T′ has been drilled is shown. In use, the surgeon attaches the second end 1b of the loop member 1 on the hook 95, whereby the tissue graft 20 extends over the loop member 1, and guides the plate 91 through the tunnel T′ by pulling on the second end 1b of the loop member 1. Once the plate 91 is outside of the tunnel T′, it can be flipped with the flip suture 19 so that it abuts on the bone surface C. In case the portion of the loop member 1 inside the tunnel T′ is too long, so that the tissue graft 20 is not sufficiently stretched (see the distance dx in FIG. 21), the surgeon may unhook the end 1b, pull thereon and attach it again on the hook 95, so that it goes through an eyelet which is arranged nearer to the end 1a. The end 1b may be shortened by cutting away the portion which is too long.

A support member according to an embodiment described above is made from a biocompatible material, e.g. metal such as titan or plastic such as PEEK (polyether ether ketone). The holding device according to the second, third and fourth embodiment may be packed such that the pieces of the support member and the loop member form loose parts, which can put together during surgery.

The loop member 1 is made as a fabric in form of a braid. A braid is a structure formed by interlacing strands of flexible material. Each strand passes alternately over and under one or more of the others. The braid may be two dimensional or three dimensional. In the latter case, a strand runs through the braid in all three directions. All or at least some of the strands of the loop member 1 extend continuously from the first end 1a to the second end 1b. In an embodiment, in which a loop member 1 has an intermediate portion which is thicker than the ends 1a, 1b (see FIG. 16), some of the strands extend from a first location to a second location, wherein the locations define the intermediate portion and are thus spaced away from the ends 1a, 1b.

The braided loop member 1 has a high strength. The strands are interlaced such that they do not get separated from each other when for instance a force acts on the location of the bifurcation or convergence of an eyelet 5a, 5b. The location of bifurcation/convergence can be reinforced e.g. if necessary with an additional thermal in line process after the braiding.

The loop member 1 may be fabricated by means of a braiding machine comprising bobbins, which carry the strands. In the braiding operation, the bobbins are advanced along specific paths of travel, see e.g. EP 2 492 385 A2. The number of bobbins may be 12 up to several dozens, e.g. 120.

The loop member 1 is made from a biocompatible material. A strand to be used for braiding includes fibers of the same or different type. Examples of suitable materials for the fibers are:
chemical fibers, e.g. ultra-high-molecular-weight polyethylene, polyester, polypropylene, polyamide
natural fibers, e.g. silk, flax, hemp
metallic fibers, e.g. nickel titanium (Nitinol)
biodegradable fibers, e.g. polylactic acid (PLA), polyglycolic acid (PGA).

It also possible to combine different types of fibers, known as fiber blends, to produce the loop member 1, e.g. polyester with polyamide, polyester with hemp, flax with silk, etc.

A fiber may be formed as a single yarn or as multiple yarns, which are twisted together or are combined without twist.

FIGS. 22 to 24 shows schematically an example of a loop member 1 including a multiple of braided strands 6. Here, the loop member 1 has a tubular structure including tubular intermediate portions 3 which bifurcate into two tubular branches 4a, 4b at an eyelet 5. Optionally, the interior 7, 7a, 7b of these formed tubes may include a core. The core may include e.g. strands, which are straight, twisted or braided. The core may be made of one or more of the following materials: textile material, non-textile material, metal, plastic, etc. The core may include e.g. a tube, in particular a plastic tube, a metal wire, a mesh, a webbing, etc.

Other braided structures for the loop member 1 are conceivable, for instance flat braids having bifurcating and converging portions to define eyelets.

Machines arranged for flat braiding cause the yarn carriers to move on a line back and forth. This results in a flat structure without an interior. Machines arranged for tubular braiding cause the yarn carriers to move in a circle. This results in a tubular structure having an interior, which may or may not include a core.

Individual components of the loop member 1, i.e. fiber, yarn, strand, core, etc., or the loop member 1 as a whole may be provided with an additional layer obtained e.g. by impregnating a substance or by plasma deposition.

The holding device has various medical applications. It can be used e.g. in the reconstruction of knee ligaments, in particular the anterior cruciate ligament and the posterior cruciate ligament. In these applications, the holding devices serve to attach a tissue graft replacing the ligament so that it has the desired strength. Another application is in the treatment of injuries of the acromioclavicular joint (AC joint), which is the joint at the top of the shoulder. In one application, the holding device is used to stabilize the clavicle to the coracoid process. To this end, the surgeon may drill a tunnel into the clavicle, loop the loop member around the coracoid process and attach it to the support member.

The advantages of the embodiments described so far can be summarized as follows:
The holding device may be configured such that the loop member only and not the support member is moved through a bone tunnel. This allows the tunnel to be made smaller in diameter, simplifies the pulling through of the loop member in particular around narrow areas, and results in increased varieties in form and dimension of the support member. In particular, the support member can be configured to be greater in the size than the bone tunnel.
The loop member can be given a desired length by choosing an appropriate eyelet at the first and/or second end for attachment to the support member. Thus, it is possible to adjust the effective length of the loop member. During operation, it is possible to release the loop member again from the support member and to change the length if necessary by choosing another eyelet. The eyelets of the loop member may also be used for handling of the holding device, e.g. by passing a suture therethrough.
The attachment between loop member and support member is relatively simple by passing a portion of an attachment element through an eyelet. No clamping nor splicing are necessary.
One implant for all various length sizes makes manufacturing as well as storage and handling at the consumer's side easier and has a higher cost effectiveness.

1. Implantable holding device for acting on bones (B, F, T), in particular for ligament reconstruction and/or stabilization of a joint but not limited to, comprising a flexible loop member (1) having a first end (1a), a second end (1b) and a plurality of eyelets (5, 5a, 5b) arranged between the first end and the second end, the loop member being braided to form bifurcating and converging portions which define the eyelets, and
a support member (11; 31, 41; 51, 61; 71, 81; 91) configured to lie against a
bone and comprising an attachment element (15; 25; 41; 61; 83; 83′; 95), the support member having a first attachment location for attachment of the first end of the loop member and a second attachment location for attaching the loop member by passing a portion of the attachment element through an eyelet.

2. The holding device according to claim 1, wherein the loop member (1) has an intermediate portion extending from a first location (1d) to a second location (1e) and having a length, which is greater than the length from the first end (1a) to the first location (1d) and/or the length from the second end (1b) to the second location (1e), wherein the intermediate portion (1c) is free of eyelets (5) and/or has a width, which is greater than the width of the first end (1a) and/or the width of the second end (1b).

3. The holding device according to claim 1, wherein the support member (11; 31, 41; 51, 61; 91) comprises a least one passage (12a, 12b; 33; 53; 92a, 92b) through which the loop member (1) can be passed.

4. The holding device according to claim 1, wherein the support member comprises a base element (11; 91) having a surface (11a; 91a) to contact the bone (B, F), the attachment element (15; 95) being made integral with the base element.

5. The holding device according to claim 1, wherein the support member (11′, 26; 31, 41; 51, 61; 71, 81; 83′) comprises a base element (11′; 31; 51; 71) to contact the bone (B, F), the attachment element (26; 41; 61; 81; 83′) being attachable to the base element.

6. The holding device according to claim 1, wherein the attachment element (41; 61; 81) comprises a least one protruding arm (44a, 44b, 64a, 64b; 83) configured to be passed through an eyelet (5, 5a, 5b).

7. The holding device according to claim 6, wherein the base element (31; 51) comprises at least one groove (34a, 34b; 54a, 54b) to receive the at least one arm (44a, 44b, 64a, 64b) of the attachment element (41; 61).

8. The holding device according to claim 1, wherein the support member (51, 61) comprises a base element (51) having a flange portion (55) and an insert portion (56) being insertable into a tunnel (T′) formed in the bone (B).

9. The holding device according to claim 1, wherein the support member comprises two base elements (71), which are preferably formed in the same way, and an attachment element (83′), which is attachable to the two base elements (71).

10. The holding device according to claim 1, made of a biocompatible material.

11. The holding device according to claim 1, wherein the loop member (1) is a tubular braid or a flat braid.

12. The holding device of claim 4, wherein the attachment element being formed as a tongue.

13. The holding device of claim 5, wherein the attachment element is attachable to the base element by means of a snap attachment

14. The holding device of claim 6, wherein the attachment element comprises two protruding arms configured to be passed through an eyelet.

15. The holding device of claim 9, wherein the attachment element is a pin, each end of the pin being receivable in a hole formed in a respective base element.

16. The holding device according to claim 2, wherein the support member (11; 31, 41; 51, 61; 91) comprises a least one passage (12a, 12b; 33; 53; 92a, 92b) through which the loop member (1) can be passed.

17. The holding device according to claim 2, wherein the support member comprises a base element (11; 91) having a surface (11a; 91a) to contact the bone (B, F), the attachment element (15; 95) being made integral with the base element.

18. The holding device according to claim 3, wherein the support member comprises a base element (11; 91) having a surface (11a; 91a) to contact the bone (B, F), the attachment element (15; 95) being made integral with the base element.

19. The holding device according to claim 2, wherein the support member (11′, 26; 31, 41; 51, 61; 71, 81; 83′) comprises a base element (11′; 31; 51; 71) to contact the bone (B, F), the attachment element (26; 41; 61; 81; 83′) being attachable to the base element.

20. The holding device according to claim 3, wherein the support member (11′, 26; 31, 41; 51, 61; 71, 81; 83′) comprises a base element (11′; 31; 51; 71) to contact the bone (B, F), the attachment element (26; 41; 61; 81; 83′) being attachable to the base element.
